(19) United States
(12) Patent Application Publication
(10) Pub. No.: US 2026/0114852 A1
(43) Pub. Date: Apr. 30, 2026

(54) DEVICE FOR GUIDING A USER IN ULTRASOUND ASSESSMENT OF AN ORGAN

(71) Applicant: DIAGNOLY, Lyon (FR)

(72) Inventors: Ivan Voznyuk, Villeurbanne (FR); Camille Dupont, Lyon (FR)

(21) Appl. No.: 19/473,457

(22) PCT Filed: Mar. 20, 2024
(86) PCT No.: PCT/EP2024/057500

§ 371 Date: Oct. 7, 2025

(30) Foreign Application Priority Data
Apr. 7, 2023 (EP) 23167214.8

(51) Int. Cl. A61B 8/00; A61B 8/08

(52) U.S. Cl. CPC A61B 8/5223; A61B 8/0833; A61B 8/0866; A61B 8/463

(57) ABSTRACT

The present invention relates to a device (1000) and method for guiding a user in assessment of an organ, said assessment being based on at least two ultrasound images (I1, . . . , In), said device comprising a processor configured for each ultrasound image, to receive a list of remarkable elements (e1, e2, e3, . . . , ek) containing information (10-16), and to output a list of first criteria (c1, c2, . . . , ci), wherein each first criterion is associated with at least two first scores (30, 40), comprising information about a defect and an impossibility to assess the first criterion, and a temporal module configured to receive the list of first criteria (c1, c2, . . . , ci) and/or the list of remarkable elements, and output a list of global criteria (C1, C2, . . . , Cj) associated with at least two global scores (70, 80) representative of the defect across the whole set of ultrasound images (I1, . . . , In).

WO

### FIELD OF INVENTION

The present invention relates to the field of medical imaging. In particular, the invention relates to a device and method for guiding a user in ultrasound assessment of an organ so as to perform a screening evaluation of said organ during a medical examination.

### BACKGROUND OF INVENTION

Ultrasound imaging is a non-invasive diagnostic technique used to image the inside of the body, using ultrasound waves. There are many uses of ultrasound imaging, including imaging of the heart, blood vessels, eyes, thyroid, brain, breast, abdominal organs, skin, and muscles.

In order to assess a specific organ, medical guidelines have defined lists of first criteria that may be verified on ultrasound images. These first criteria are indicative of an organ having a defect. Physicians may derive their diagnosis from the verification of the first criteria in at least one ultrasound image.

Such first criteria may be verified by analyzing remarkable elements present in the ultrasound images. A remarkable element is an ultrasound structural feature of the ultrasound image that contains information that may directly or indirectly be used to verify a first criterion.

One of the most common uses of ultrasound is during pregnancy, to monitor the growth and development of the fetus by detecting fetal abnormalities. A fetal abnormality is a defect in the fetus of a genetic and/or structural nature that can cause significant fetal health and developmental complications, maternal complicated delivery and lead to complex postnatal medical interventions and adverse outcomes.

Detection of fetal anomalies allows to anticipate a tailored inborn birth, prompt medical care or surgical treatment in specialized centers at birth or in the neonatal period, as well as to provide an adequate information to the parents. For all of these reasons, the ability to detect fetal complications prenatally is critical.

However, despite the progress in equipment, up to 52.8% of prenatal cardiac pathologies are unfortunately not detected today. This high rate is due to the fact that the examination is highly “operator-dependent” as well as a “patient-dependent”. As a result, these factors can generate errors in prenatal diagnosis and an overload of work involving a lack of time for health care staff.

According to specialists, fetal heart disease is not always screened antenatally for 3 main reasons: (1) in 49% of cases, lack of skill in adapting ultrasound images (the ultrasound images obtained by the operator are not sufficient to make the correct diagnosis); (2) in 31% of the cases, lack of experience in diagnostics (the images obtained are good and the prenatal pathology is visible but not recognized by the operator); and finally (3), in 20% of cases, pathologies cannot be detected because they are not visible on the ultrasound images.

Different computer-implemented methods have been proposed to help the users to assess ultrasound images of organs and analyze these images in order to detect a large spectrum of abnormalities. More precisely, these methods may be configured to process each image independently to predict a probability of an organ having at least one abnormality related to a predetermined first criterion.

For instance, a binary system may be configured to detect as a remarkable element in a given ultrasound image a blood vessel in a specific position with respect to the heart. The first criterion to be verified is the absence of said blood vessel at this specific position. Indeed, if this blood vessel is detected at this specific position, it means that there is a malformation that might cause symptoms such as high blood pressure in the right side of the heart. If the ultrasound image contains an artifact that makes the area where the blood vessel is supposed to be detected impossible to analyze, the system may output a high probability of absence of the blood vessel because it interprets the artifact as the absence of said blood vessel when in facts it is hidden by the artifact. In the end, the binary system will output an unreliable result because its decision is based on a corrupted image.

As another example, a binary system may be configured to detect as a remarkable element in a given ultrasound image a discontinuity in the heart interventricular septum. The first criterion to be verified is a septal defect. Indeed, a hole in the septum causes an increased amount of blood that flows toward the lungs. Over time, it may cause damage to the blood vessels in the lungs and ultimately lead to high blood pressure and heart failure. However, a vertical angle between the probe and the septum may cause a dropout of echoes throughout the septum, hence forming a shadow resembling the appearance of hole in the septum, even in normal hearts. The binary system interpretation of the septum defect in these viewpoint conditions may lead to overdiagnosis of interventricular septal defects.

In all of the examples above, the system relies on the analysis of individual ultrasound images. Therefore, some prediction may be biased due to an unsuitable viewpoint of the ultrasound image. Moreover, ultrasound images represent the organ state at a specific time point. Therefore, these systems are unable to assess temporal criteria, such as the regular opening and closing of a valve. Additionally, such systems are not configured to reflect on the accuracy of their prediction. Therefore, the physician has no way to verify the accuracy of the system deduction unless he analyzes himself the ultrasound image. This lack of reliability may cause a huge time loss or even worse a wrong diagnosis from the physician.

Therefore, the problem solved by the present invention is to provide a device and a method with an improved prediction accuracy and a greater robustness with respect to errors prediction and assessment of temporal criteria.

### SUMMARY

The present invention related to a device for guiding a user in ultrasound assessment of an organ to perform an evaluation of said organ during a medical examination, said ultrasound assessment being based on at least two ultrasound images resulting from at least a partial reflection of ultrasound waves by said organ, said device comprising at least one processor configured to:
for each ultrasound image:
   receive as an input, for each ultrasound image of the at least two ultrasound images, a list of remarkable elements associated to the ultrasound image, wherein each remarkable element is associated with at least one remarkable information among:
      an observability of the remarkable element or an unobservability of the remarkable element,
      an abnormal aspect of the remarkable element or a non-abnormal aspect of the remarkable element,
      a normal aspect of the remarkable element or a non-normal aspect of the remarkable element, and
      a measurement performed on the remarkable element,
   generate and output a list of first criteria indicative of a health status of the organ, wherein each first criterion of the list of first criteria is associated with at least two first scores, each first score being generated using said at least one remarkable information associated to the at least one remarkable element, said at least two first scores being such that a combination of the at least two first scores provides information on an impossibility to assess the first criterion, a presence of a defect and an absence of a defect,
generate and output a list of global criteria indicative of a health status of the organ assessed over said at least two ultrasound images, wherein each global criterion of the list of global criteria is associated with at least two global scores, said at least two global scores being such that a combination of the at least two global scores provides an information on a global impossibility to assess the global criterion, a global presence of a defect and a global absence of a defect, wherein the list of global criteria is generated using a feed-forward network of arithmetic operators of linear or non-linear nature which maps at least two of said generated lists of first criteria, and/or at least two lists of remarkable elements relative to the at least two ultrasound images from which said at least two lists of first criteria have been generated, to the at least two global scores.

According to the invention, a health status of the organ corresponds to a presence or an absence of a defect in the organ.

A first advantage of the device of the invention concerns the nature of the first scores and global scores. Indeed, the first scores and global scores respectively convey an impossibility to assess the first criterion and global criterion. This can help improving the accuracy of the assessments by reducing errors made by the system. If an image is impossible to assess, it can be flagged for review, and the presence or absence of a defect result can be double-checked to ensure its accuracy. If the whole set of ultrasound images is impossible to assess, the system can notify the physician that he needs to acquire more ultrasound images.

As a result, the device only provides predictions when it has enough information to do so with a high level of confidence. When it encounters data that is too ambiguous or incomplete, it will simply say “I do not know” rather than making a guess. This reduces the likelihood of false positives or false negatives and ensures that healthcare professionals can rely on the algorithm's predictions with a high degree of confidence.

Therefore, the combination of three information in the first scores and global scores can improve the efficiency of the diagnostic process. By automating the detection process and the calculation of the scores, physicians can save time and resources when evaluating images. They can be more confident in the results outputted by the device without having to double-check the ultrasound images analysis.

Another advantage of the invention relates to the global scores. Indeed, they have a reduced error rate compared to first scores calculated on individual ultrasound images. The global scores provide a summary of the three types of global information across the whole set of ultrasound images. As a result, the system is more robust with respect to prediction errors. Even when faced with imperfect data, such as ultrasound images of poor quality, it can still produce reliable results.

According to one embodiment, at least one global criterion is a temporal criterion indicative of a defect in a periodic evolution of the organ and the at least two global scores are determined based on the evolution in time of the at least one remarkable element across the at least two ultrasound images.

This temporal criterion is particularly advantageous for the assessment of organs that, for their nature, modify periodically their configuration such as the heart, the lungs, and the like. More precisely, the temporal criterion is particularly advantageous to verify if a valve is regularly opening and closing, which is impossible to verify in isolated ultrasound images.

According to one embodiment, each first criterion of the list of first criteria is further associated with a first severity score representative of a magnitude of the defect.

By quantifying the severity of the abnormal aspect, the severity score can provide additional information to the physician that may not be immediately apparent from a visual inspection of the ultrasound image. A high severity score will catch more the attention of the physician and make him more attentive during his analysis of the corresponding ultrasound image.

According to one embodiment, each global criterion of the list of global criteria is further associated with a global severity score representative of a magnitude of the defect assessed on the at least two ultrasound images.

A global severity score can help to more accurately identify and diagnose abnormalities or pathologies within the organ. Advantageously, by quantifying the severity of the abnormality, the global severity score can provide additional information that may not have been detected by the physician during his own analysis of the ultrasound images.

According to one embodiment, the at least one remarkable information is comprised in at least one remarkable score.

According to one embodiment, each first criterion of the list of first criteria is associated with three mutually exclusive first scores, including:
an assessability score, representative of the impossibility to assess the first criterion,
a presence score, representative of the presence of a defect, and
an absence score, representative of the absence of a defect.

The first scores outputted by the transformation module may be available for the physician to consult. These first scores provide a summary of the three types of first information and can help the physician verifying and/or deciding on the assessment of a criterion on a given ultrasound image. Indeed, advantageously the first scores can make the ultrasound data more accessible and easier to interpret even for non-expert users, thus improving its usefulness in decision-making.

Defining one first information per first score is the easiest way to convey the first information to the physician. He only needs to consider the three first scores independently to draw his conclusions on a specific ultrasound image. The presence and absence scores, may be interpreted by the physician so as to evaluate the probability of presence or absence of a defect in the organ on a given ultrasound image based on the remarkable elements analyzed for this criterion. The assessability score may be interpreted by the physician so as to evaluate the sufficiency or insufficiency of the remarkable information associated with the at least one remarkable element used to assess the criterion. In other words, the assessability score may be used to double check the correctness of the presence and absence scores. Indeed, a first score conveying a high probability of presence or absence of a defect combined with an assessability score conveying an impossibility to assess the criterion is unreliable and should not be taken into account by the physician to draw his diagnosis.

According to one embodiment, each first criterion of the list of first criteria is associated with two first scores:
a defect score representative of the presence of a defect and the absence of a defect, said defect score being comprised between a lower score bound and an upper score bound, the lower score bound being representative of a high probability of an absence of a defect and the upper score bound being representative of a high probability of a presence of a defect, and
an assessability score, being representative of the impossibility to assess the first criterion.

According to one embodiment, each global criterion of the list of global criteria is associated with two global scores:
a global defect score representative of the global absence of a defect and the global presence of a defect, said global defect score being comprised between a global lower score bound and a global upper score bound, the global lower score bound being representative of a high probability of an absence of a defect across the at least two ultrasound images and the upper score bound being representative of a high probability of a presence of a defect across the at least two ultrasound images, and
a global assessability score, representative of the global impossibility to assess the global criterion.

According to one embodiment, the generation of the list of global criteria comprises implementing a 1-dimensional convolutional neural network configured to simultaneously process the outputted lists of first criteria and/or the lists of remarkable elements relative to the at least two ultrasound images.

According to one embodiment, the generation of the list of global criteria comprises implementing a recurrent convolutional neural network configured to iteratively process the outputted lists of first criteria and/or the lists of remarkable elements relative to the at least two ultrasound images.

There are several advantages to giving first scores as an input to a convolutional neural network (CNN) instead of directly giving raw data. Indeed, first scores represent a compact summary of complex raw data, thus reducing the dimensionality of the input. This helps to reduce the number of parameters in the CNN and improve its computational efficiency. Moreover, the first scores are less sensitive to noise or variations that may be present in the raw data, as they represent a more robust and generalized representation of the underlying first information. Furthermore, the first scores can be more easily transferred across different datasets or applications, as they often represent a more generalizable representation of the underlying first information that is less dependent on the specific characteristics of the input data.

As a result, the system is extremely light in terms of memory, making it easy to use on a wide range of devices.

According to another aspect, the invention relates to a computer-implemented method for guiding a user in ultrasound assessment of an organ to perform a diagnosis or screening evaluation of said organ during a medical examination, said ultrasound assessment being based on at least two ultrasound image resulting from at least a partial reflection of ultrasound waves by said organ, said method comprising:
for each ultrasound image:
   receiving a list of remarkable elements associated to the at least two ultrasound images, wherein each remarkable element is associated with at least one remarkable information among:
      an observability of the remarkable element or an unobservability of the remarkable element,
      an abnormal aspect of the remarkable element or a non-abnormal aspect of the remarkable element,
      a normal aspect of the remarkable element or a non-normal aspect of the remarkable element, and
      a measurement performed on the remarkable element,
   generating and outputting a list of first criteria indicative of a health status of the organ, wherein each first criterion of the list of first criteria is associated with at least two first scores, each first score being generated using said at least one remarkable information associated to the at least one remarkable element, said at least two first scores being such that a combination of the at least two first scores provides information on an impossibility to assess the first criterion, a presence of a defect, and an absence of a defect,
generating and outputting a list of global criteria indicative of a health status of the organ assessed over said at least two ultrasound images, wherein each global criterion of the list of global criteria, said at least two global scores being such that a combination of the at least two global scores provides information on a global impossibility to assess the global criterion, a global presence of a defect and a global absence of a defect, wherein the list of global criteria is generated using a feed-forward network of arithmetic operators of linear or non-linear nature which maps at least two of said generated lists of first criteria, and/or at least two lists of remarkable elements relative to the at least two ultrasound images from which said at least two lists of first criteria have been generated, to the at least two global scores.

According to another aspect, the invention relates to a computer program product for guiding a user in ultrasound assessment of an organ, the computer program product comprising instructions which, when the program is executed by a computer, cause the computer to automatically carry out the method described previously.

According to another aspect, the invention relates to a computer-readable storage medium comprising instructions which, when executed by a computer, cause the computer to carry out the method for guiding a user in ultrasound assessment of an organ as described previously.

### BRIEF DESCRIPTION OF THE DRAWINGS

FIG. 1 is a block diagram representation of the device according to an embodiment of the invention,

FIG. 2 is an ultrasound image comprising abnormal remarkable elements analyzed for the fourth vessel criterion and the remarkable scores of the remarkable elements,

FIG. 3 is an ultrasound image comprising normal remarkable elements analyzed for the fourth vessel criterion and the remarkable scores of the remarkable elements,

FIG. 4 is an ultrasound image comprising inobservable remarkable elements analyzed for the fourth vessel criterion and the remarkable scores of the remarkable elements,

FIG. 5 is an ultrasound image comprising abnormal and non-normal remarkable elements analyzed for the septum defect criterion and the remarkable scores of the remarkable elements,

FIG. 6 is an ultrasound image comprising normal and non-abnormal remarkable elements analyzed for the septum defect first criterion and the remarkable scores of the remarkable elements,

FIG. 7 is an ultrasound image comprising unobservable remarkable elements analyzed for the septum defect first criterion and the remarkable scores of the remarkable elements,

FIG. 8 is an ultrasound image comprising two measurement remarkable elements analyzed for ventricles asymmetry first criterion and the values of the measurement performed on the remarkable elements, wherein there is an asymmetry,

FIG. 9 is an ultrasound image comprising two measurement remarkable elements analyzed for ventricles asymmetry first criterion and the remarkable scores of the remarkable elements, wherein there is no asymmetry,

FIG. 10 is an ultrasound image comprising two measurement remarkable elements analyzed for ventricles asymmetry first criterion and the remarkable scores of the remarkable elements, wherein one remarkable element is unobservable,

FIG. 11 is an ultrasound image comprising two measurement remarkable elements analyzed for ventricles asymmetry first criterion and the remarkable scores of the remarkable elements, wherein there is a bigger asymmetry than in FIG. 8,

FIG. 12 is a representation of three mutually exclusive first scores outputted by the transformation module in relation with the ultrasound image from FIG. 2, respectively a presence score, an absence score and an assessability score,

FIG. 13 is a representation of three mutually exclusive first scores outputted by the transformation module in relation with the ultrasound image from FIG. 3, respectively a presence score, an absence score and an assessability score,

FIG. 14 is a representation of three mutually exclusive first scores outputted by the transformation module in relation with the ultrasound image from FIG. 4, respectively a presence score, an absence score and an assessability score,

FIG. 15 is a representation of a second embodiment of two first scores outputted by the transformation module in relation with the ultrasound image from FIG. 5, respectively an assessability score and a defect score,

FIG. 16 is a representation of a second embodiment of two first scores outputted by the transformation module in relation with the ultrasound image from FIG. 6, respectively an assessability score and a defect score,

FIG. 17 is a representation of a second embodiment of two first scores outputted by the transformation module in relation with the ultrasound image from FIG. 7, respectively an assessability score and a defect score,

FIG. 18 is a representation of three mutually exclusive first scores and a severity score outputted by the transformation module in relation with the ultrasound image from FIG. 8,

FIG. 19 is a representation of three mutually exclusive first scores and a severity score outputted by the transformation module in relation with the ultrasound image from FIG. 9,

FIG. 20 is a representation of three mutually exclusive first scores and a severity score outputted by the transformation module in relation with the ultrasound image from FIG. 10,

FIG. 21 is a representation of three mutually exclusive first scores and a severity score outputted by the transformation module in relation with the ultrasound image from FIG. 11,

FIG. 22 is an ultrasound image at a first time point comprising two measurement remarkable elements analyzed for ventricles asymmetry first criterion, the remarkable scores of the remarkable elements, and three mutually exclusive first scores associated with the first criterion wherein an asymmetry is observed,

FIG. 23 is an ultrasound image at a second time point comprising two measurement remarkable elements analyzed for ventricles asymmetry first criterion, the remarkable scores of the remarkable elements, and three mutually exclusive first scores associated with the first criterion wherein an asymmetry is observed,

FIG. 24 is an ultrasound image at a third time point comprising two measurement remarkable elements analyzed for ventricles asymmetry first criterion, the remarkable scores of the remarkable elements, and three mutually exclusive first scores associated with the first criterion wherein an asymmetry is observed,

FIG. 25 is an ultrasound image at a first time point comprising two measurement performed on remarkable elements analyzed for ventricles asymmetry first criterion, the remarkable scores of the remarkable elements, and three mutually exclusive first scores associated with the first criterion wherein no asymmetry is observed,

FIG. 26 is an ultrasound image at a second time point comprising two measurement performed on remarkable elements analyzed for ventricles asymmetry first criterion, the remarkable scores of the remarkable elements, and three mutually exclusive first scores associated with the first criterion wherein an asymmetry is observed,

FIG. 27 is an ultrasound image at a third time point comprising two measurement performed on remarkable elements analyzed for ventricles asymmetry criterion, the remarkable scores of the remarkable elements, and three mutually exclusive first scores associated with the first criterion wherein no asymmetry is observed,

FIG. 28 is an ultrasound image at a first time point comprising two measurement performed on remarkable elements analyzed for ventricles asymmetry first criterion, the remarkable scores of the remarkable elements, and three mutually exclusive first scores associated with the first criterion wherein the right ventricle is unobservable,

FIG. 29 is an ultrasound image at a second time point comprising two measurement performed on remarkable elements analyzed for ventricles asymmetry first criterion, the remarkable scores of the remarkable elements, and three mutually exclusive first scores associated with the first criterion wherein the right ventricle is unobservable,

FIG. 30 is an ultrasound image at a third time point comprising two measurement performed on remarkable elements analyzed for ventricles asymmetry first criterion, the remarkable scores of the remarkable elements, and three mutually exclusive first scores associated with the first criterion wherein the right ventricle is unobservable,

FIG. 31 is an ultrasound image at a first time point comprising one remarkable element analyzed for static mitral valve global criterion, wherein the valve is observable and closed,

FIG. 32 is an ultrasound image at a second time point comprising one remarkable element analyzed for static mitral valve global criterion, wherein the valve is observable and closed,

FIG. 33 is an ultrasound image at a third time point comprising one remarkable element analyzed for static mitral valve global criterion, wherein the valve is observable and closed,

FIG. 34 is an ultrasound image at a first time point comprising one remarkable element analyzed for static mitral valve global criterion, wherein the valve is observable and closed,

FIG. 35 is an ultrasound image at a first time point comprising one remarkable element analyzed for static mitral valve global criterion, wherein the valve is observable and openened,

FIG. 36 is an ultrasound image at a first time point comprising one remarkable element analyzed for static mitral valve global criterion, wherein the valve is observable and closed,

FIG. 37 is a representation of three mutually exclusive global scores outputted by the temporal module in relation with the ultrasound images from FIGS. 22 to 24,

FIG. 38 is a representation of three mutually exclusive global scores outputted by the temporal module in relation with the ultrasound images from FIGS. 25 to 27,

FIG. 39 is a representation of three mutually exclusive global scores outputted by the temporal module in relation with the ultrasound images from FIGS. 28 to 30,

FIG. 40 is a representation of two global scores outputted by the temporal module in relation with the ultrasound images from FIGS. 31 to 33,

FIG. 41 is a representation of two global scores outputted by the temporal module in relation with the ultrasound images from FIGS. 34 to 36,

FIG. 42 is a representation of a matrix that may be fed as an input to the temporal module according to an embodiment,

FIG. 43 is a representation of a matrix that may be outputted by the temporal module according to an embodiment,

FIG. 44 is a flow chart showing successive steps executed with the device for guiding of FIG. 1,

FIG. 45 diagrammatically shows an apparatus, comprising at least one graphic processor, integrating the functions of the device for guiding of FIG. 1, and

FIG. 46 diagrammatically shows an apparatus, comprising only CPU, integrating the functions of the device for guiding of FIG. 1, according to a specific embodiment.

### DETAILED DESCRIPTION

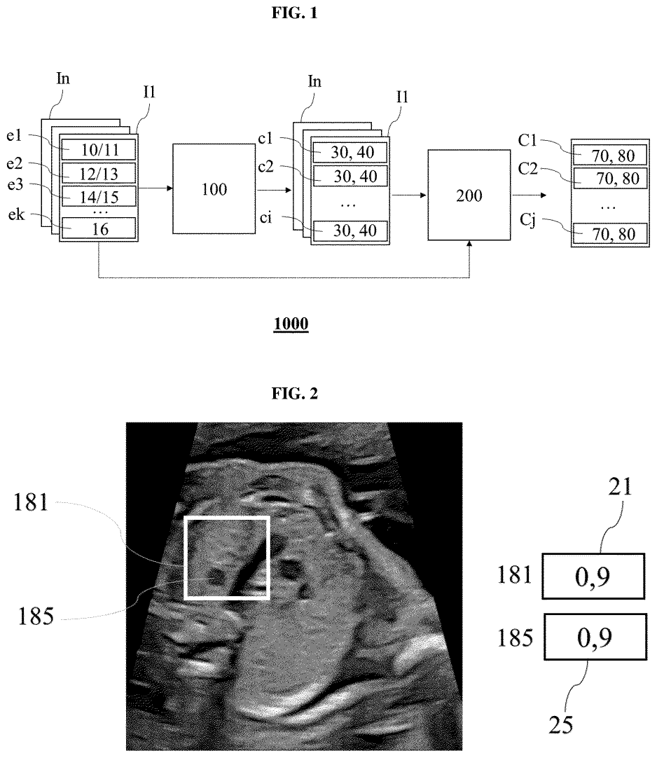

The present disclosure will be described in reference to different embodiments of a device 1000 for guiding a user in ultrasound assessment of an organ, as illustrated on FIG. 1.

During the years, medical guidelines have defined first criteria c1, c2, . . . , ci, that is ultrasound findings indicative of an organ health status that may be used by the physician to diagnose a disease. For instance, a first criterion may be a septal defect, a mass in the left ventricle, or a ventricular asymmetry. Such first criteria c1, c2, . . . , ci may be verified based on the analysis of at least one remarkable element e1, e2, e3, . . . , ek present in an ultrasound image I1, . . . , In. According to the invention, a remarkable element e1, e2, e3, . . . , ek refers to an ultrasound structural feature associated with at least one remarkable information among at least four types of remarkable information. Advantageously, the at least one information associated with each remarkable element e1, e2, e3, . . . , ek may be comprised in at least one score.

The device 1000 may comprise a transformation module 100 that receives as input n lists of k remarkable elements e1, e2, e3, . . . , ek. A list of remarkable elements e1, e2, e3, . . . , ek may be extracted from an ultrasound image I1, . . . , In among a set of n ultrasound images I1, . . . , In of the organ. The number of remarkable elements e1, e2, e3, . . . , ek that may be extracted may differ depending on the ultrasound image I1, . . . , In. The remarkable elements e1, e2, e3, . . . , ek may be extracted based on a predetermined database of remarkable elements e1, e2, e3, . . . , ek, for instance using classifiers, such as described in patent applications WO2023031438 and EP20305972.

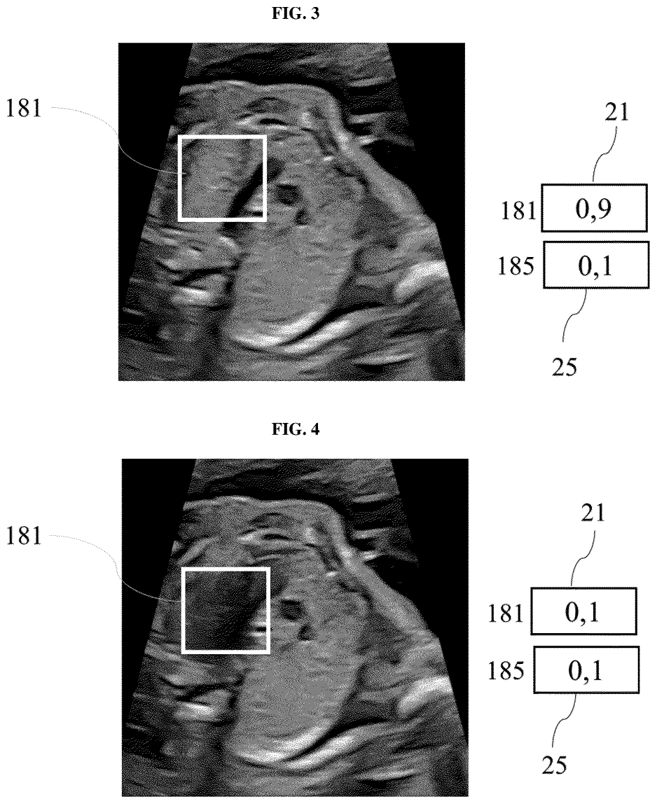

The first type of remarkable information associated to one remarkable element e1, e2, e3, . . . , ek relates to an observability 10 or an unobservability 11 of the remarkable element e1, e2, e3, . . . , ek on a given ultrasound image. The unobservability of the remarkable element may either be caused by a real absence of the remarkable element in the ultrasound image or by an impossibility to detect the remarkable element because of a wrong angle or because of the presence of an artifact ultrasound shadow in the ultrasound image. For instance, in FIGS. 2 to 4, a remarkable element 181 that may be extracted is the region beside the ductal arch. In FIGS. 2 and 3, the region is well observable and the remarkable element 181 can be extracted. However, in FIG. 4, the region is hidden by a shadow and the remarkable element 181 cannot be correctly extracted because the outlines of the region cannot be determined. Therefore, the remarkable information associated with the remarkable element 181 in FIGS. 2 and 3 is a remarkable information of observability 10. On the contrary, in FIG. 4, the remarkable information associated with the remarkable element 181 is a remarkable information of unobservability 11. To convey the remarkable information 10, 11, it is possible to provide a remarkable score 21 as an input to the transformation module 100. Such remarkable score 21 may be comprised between 0 and 1, a remarkable score approaching 0 conveying an unobservability 11 and a remarkable score approaching 1 conveying an observability 10. For instance, in FIGS. 2 and 3, the remarkable score 21 is equal to 0.9. On the contrary, in FIG. 4, the remarkable score 21 is equal to 0.1.

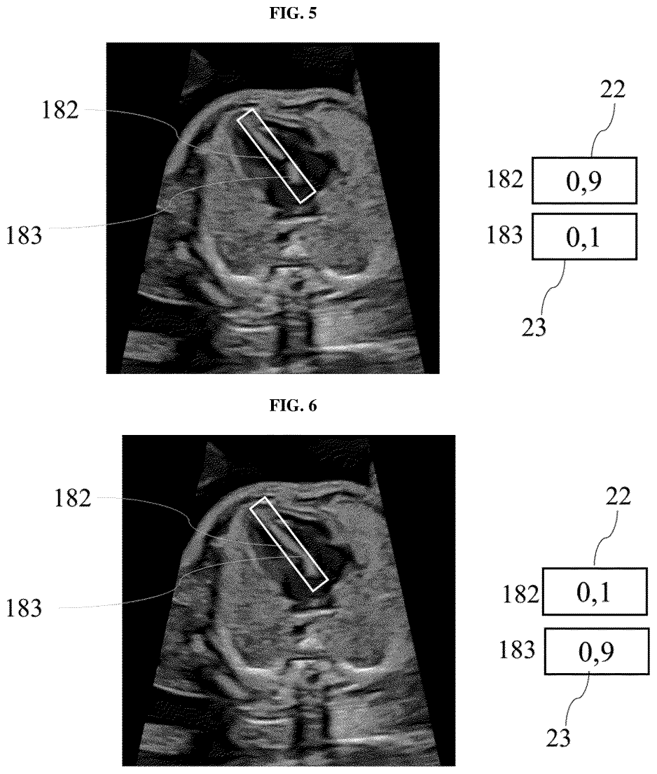

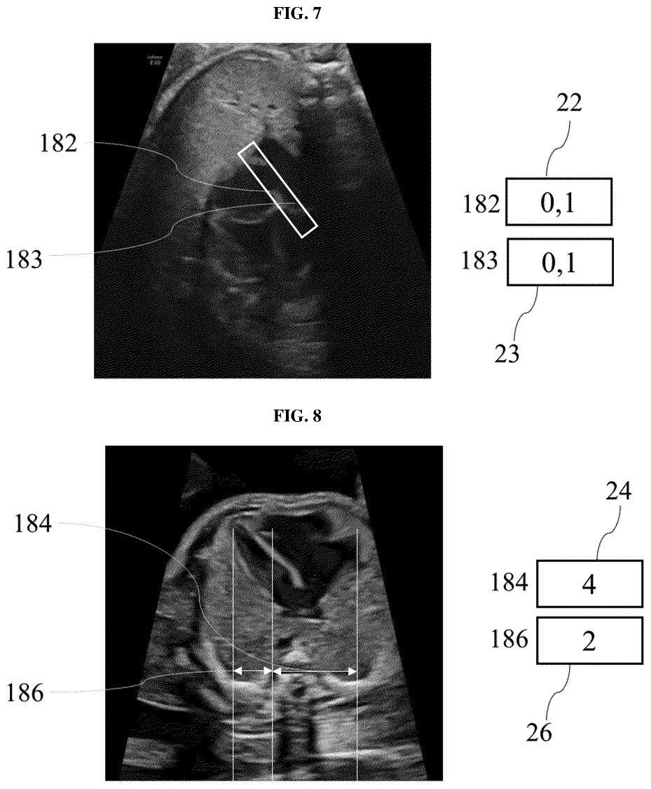

The second type of remarkable information associated to one remarkable element e1, e2, e3, . . . , ek relates to an abnormal aspect 12 of the remarkable element or a non-abnormal aspect 13 of the remarkable element in a given ultrasound image. In that case, the remarkable element is a structural element or feature that is consistently present in abnormal organs. For instance, in FIGS. 5 to 7, a remarkable element 182 that may be extracted is a discontinuous septum. In FIG. 5, a discontinuous septum can be extracted. However, in FIG. 6, the septum is continuous, therefore a discontinuous septum cannot be extracted. In FIG. 7, the septum is unobservable, therefore a discontinuous septum cannot be extracted either. Hence, the remarkable information associated with the remarkable element 182 in FIG. 5 is an information of abnormal aspect 12. On the contrary, in FIG. 6, the remarkable information associated with the remarkable element 182 is a remarkable information of non-abnormal aspect 13. In FIG. 7, the remarkable information associated with the remarkable element 182 is also a remarkable information of non-abnormal aspect 13 even though the septum is unobservable. Indeed, the extraction of the remarkable element is binary: either it is detected or not. To convey the remarkable information 12, 13, it is possible to give a remarkable score 22 as an input to the transformation module 100. Such remarkable score 22 may be comprised between 0 and 1, a remarkable score approaching 1 conveying an abnormal aspect 12 and a remarkable score approaching 0 conveying a non-abnormal aspect 13. For instance, in FIGS. 6 and 7, the remarkable score 22 is equal to 0.1. On the contrary, in FIG. 5, the remarkable score 22 is equal to 0.9.

As another example given in FIGS. 2 to 4, a remarkable element 185 that may be extracted is a vessel present beside the ductal arch. In FIG. 2, a vessel can be extracted. However, in FIG. 3, no vessel is present, therefore a vessel cannot be extracted. In FIG. 4, the region is unobservable, therefore a vessel cannot be extracted either. Hence, the remarkable information associated with the remarkable element 185 in FIG. 2 is a remarkable information of abnormal aspect 12. On the contrary, in FIG. 3, the remarkable information associated with the remarkable element 185 is a remarkable information of non-abnormal aspect 13. In FIG. 7, the remarkable information associated with the remarkable element 182 is also a remarkable information of non-abnormal aspect 13. To convey the remarkable information 12, 13, a remarkable score 25 is given as an input to the transformation module 100. Such remarkable score 25 may be comprised between 0 and 1, a remarkable score approaching 1 conveying an abnormal aspect 12 and a remarkable score approaching 0 conveying a non-abnormal aspect 13. For instance, in FIGS. 3 and 4, the remarkable score 22 is equal to 0.1. On the contrary, in FIG. 2, the remarkable score 22 is equal to 0.9.

The effective detection of the remarkable element 182 in the ultrasound image may also convey a remarkable information of observability 10. Indeed, if the remarkable element is detected, it has both an abnormal aspect 12 and it is observable 10.

The third type of remarkable information associated to one remarkable element e1, e2, e3, . . . , ek relates to a normal aspect 14 of the remarkable element or a non-normal aspect 15 of the remarkable element in a given ultrasound image. In that case, the remarkable element is a structural element or feature that is consistently present in normal organs. For instance, in FIGS. 5 to 7, a remarkable element 183 that may be extracted is a continuous septum. In FIG. 6, a continuous septum can be extracted. However, in FIG. 5, the septum is discontinuous, therefore a continuous septum cannot be extracted. In FIG. 7, the septum is unobservable, therefore a continuous septum cannot be extracted either. Hence, the remarkable information associated with the remarkable element 183 in FIG. 6 is a remarkable information of normal aspect 14. On the contrary, in FIG. 5, the remarkable information associated with the remarkable element 183 is a remarkable information of non-normal aspect 15. In FIG. 7, the remarkable information associated with the remarkable element 183 is also a remarkable information of non-normal aspect 15 even though the septum is unobservable. Indeed, the extraction of the remarkable element is binary: either it is detected or not. To convey the remarkable information 14, 15, it is possible to give a remarkable score 23 as an input to the transformation module 100. Such remarkable score 23 may be comprised between 0 and 1, a remarkable score approaching 1 conveying a normal aspect 14 and a remarkable score approaching 0 conveying a non-normal aspect 15. For instance, in FIGS. 5 and 7, the remarkable score 23 is equal to 0.1. On the contrary, in FIG. 6, the remarkable score 23 is equal to 0.9.

A remarkable element may be associated with several types of information. For instance, the effective detection of the remarkable element 183 in the ultrasound image may also convey a remarkable information of observability 10. Indeed, if the remarkable element 183 is detected, it has both a normal and aspect 14 and it is observable 10.

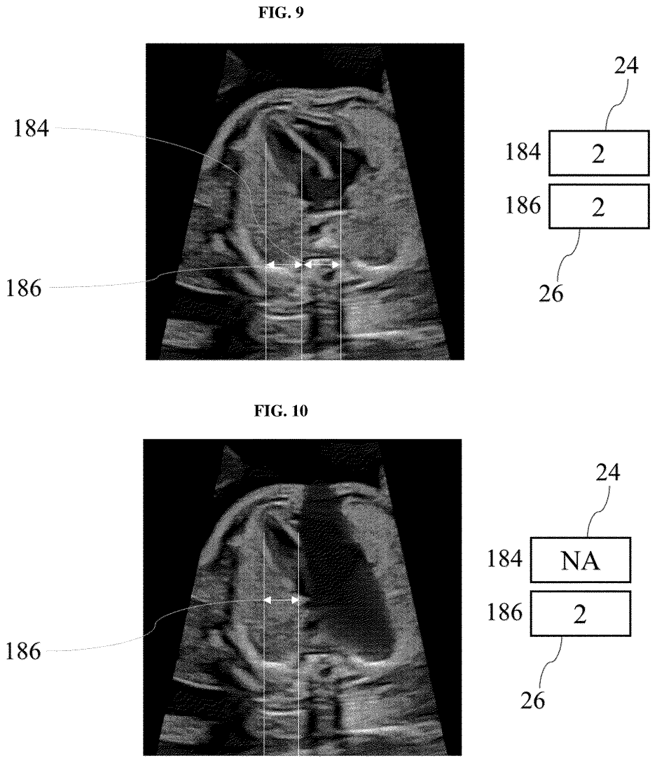

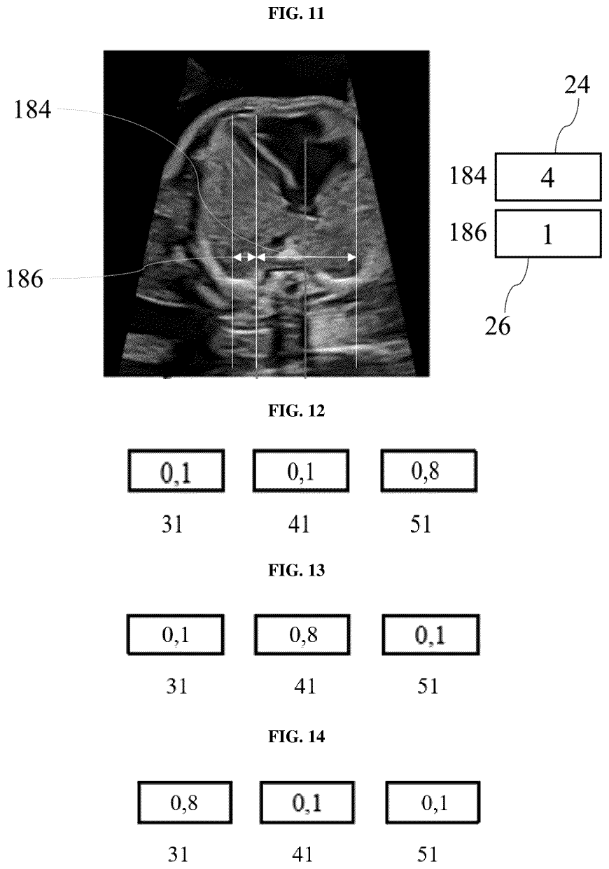

The fourth type of remarkable information associated to one remarkable element e1, e2, e3, . . . , ek relates to a measurement 16 performed on the remarkable element such as the measurement of a breadth or an orientation of the remarkable element. For instance, in FIGS. 8 to 11, two remarkable elements 184 and 186 may be extracted: the breadth of the left ventricle and the breadth of the right ventricle. These measurements 16 may be provided as an input to the transformation module 100 and may be used to verify a criterion such as the symmetry between the left and right ventricles. For instance, in FIG. 8, the breadth of the left ventricle 184 is equal to 1 and the breadth of the right ventricle 186 is equal to 4. In FIG. 11, the breadth of the left ventricle 184 is equal to 2 and the breadth of the right ventricle 186 is equal to 4. In FIG. 9, the breadth of the left ventricle 184 is equal to 2 and the breadth of the right ventricle 186 is equal to 2. In FIG. 10, the breadth of the left ventricle 184 is equal to 2 and the breadth of the right ventricle 186 is non determinable and a NA value is given as an input to the transformation module 100. Other types of information may be associated to a remarkable element e1, e2, e3, . . . , ek such as coordinates.

As illustrated in FIG. 1, the transformation module 100 is configured, for each criterion of a list of first criteria c1, c2, . . . , ci, to convert the remarkable information present in at least one remarkable elements e1, e2, e3, . . . , ek into at least two first scores 30, 40. Each first score 30, 40 conveys at least one first information among a presence of a defect, an absence of a defect and an impossibility to assess the criterion. The first score 30, 40 may be binary scores, thus conveying either that “there is a defect”, “there is no defect” or “it is impossible to assess the criterion”, and the like. The scores may also be probabilities, thus conveying a probability of presence of a defect, a probability of impossibility to assess the criterion and/or a probability of absence of a defect.

The first scores 30, 40 may be of different types as long as the outputted first scores 30, 40 convey the three types of first information when combined.

The presence of a defect may be determined based on an observability and an abnormal aspect or a non-normal aspect of at least one remarkable element. Alternatively, it may be determined based on an observability of at least one first remarkable element, and an abnormal aspect or a non-normal aspect of at least one second remarkable element. It may also be determined based on an abnormal aspect of at least one first remarkable element and a non-normal aspect of at least one second remarkable element. The absence of a defect information may be determined based on an observability of at least one first remarkable element and a non-abnormal aspect of at least one second remarkable element. It may also be determined based on a non-abnormal aspect of at least one first remarkable element and a normal aspect of at least one second remarkable element. Alternatively, it may be determined based on a normal aspect or a non-abnormal aspect of at least one remarkable element. The presence or the absence of a defect information may be determined based on a comparison of a measurement (i.e., fourth type of information) performed on a first remarkable element with respect to a reference measurement or with a measurement performed on a second remarkable element with respect to a second reference measurement. The impossibility to assess a criterion information may be determined based on an unobservability of at least one remarkable element. The impossibility to assess a criterion information may also be determined based on an unobservability of at least one first remarkable element and a non-abnormal aspect of at least one second remarkable element. Alternatively, the impossibility to assess a criterion may be determined based on a non-abnormal aspect of at least one first remarkable element and a non-normal aspect of at least one second remarkable element.

For instance, a criterion c1, c2, . . . , ci may be the fourth vessel beside the ductal arch. Such criterion c1, c2, . . . , ci may be evaluated based on two remarkable elements. The first remarkable element is the remarkable element 181 that relates to the observability 10 or unobservability 11 of the region beside the ductal arch. The second remarkable element is the remarkable element 185 that relates to the abnormal presence 12 or non-abnormal presence 13 of a vessel in the region beside the ductal arch.

By evaluating the remarkable information associated with the two remarkable elements 181, 185, it is possible to conclude on the three first information related to the criterion c1, c2, . . . , ci. For instance, in a given ultrasound image I1, . . . , In, if the remarkable element 181 gives a remarkable information of observability 10 of the region beside the ductal arch and the remarkable element 185 gives a remarkable information of abnormal aspect 12 related to the presence of a vessel in the region beside the ductal arch, it is possible to conclude to a presence of a defect in the given ultrasound image I1, . . . , In.

Alternatively, if the remarkable element 181 gives a remarkable information of observability 10 of the region beside the ductal arch and the remarkable element 185 gives a remarkable information of non-abnormal aspect 13 related to the absence of a vessel in the region beside the ductal arch, it is possible to conclude to an absence of a defect in the given ultrasound image I1, . . . , In.

Lastly, if the remarkable element 181 gives a remarkable information of unobservability 10 of the region beside the ductal arch and the remarkable element 185 gives a remarkable information of non-abnormal aspect 13 related to the absence of a vessel in the region beside the ductal arch, it is possible to conclude to an impossibility to assess the criterion c1, c2, . . . , ci in the given ultrasound image I1, . . . , In.

As another example, if the criterion c1, c2, . . . , ci is a septal defect, it may be evaluated based on two remarkable elements. The first remarkable element is the remarkable element 183 that relates to the normal aspect 14 or non-normal aspect 15 of the septum. The second remarkable element is the remarkable element 182 that relates to the abnormal aspect 12 or non-abnormal aspect 13 of the septum.

If the remarkable element 182 gives a remarkable information of abnormal aspect 12 related to a discontinuous septum and the remarkable element 183 gives a remarkable information of non-normal aspect 15 also related to a non-continuous septum, it is possible to conclude to a presence of a defect in the given ultrasound image I1, . . . , In.

If the remarkable element 182 gives a remarkable information of non-abnormal aspect 13 related to a non-discontinuous septum and the remarkable element 183 gives a remarkable information of normal aspect 14 also related to a continuous septum, it is possible to conclude to an absence of a defect in the given ultrasound image I1, . . . , In.

Lastly, if the remarkable element 182 gives a remarkable information of non-abnormal aspect 13 and the remarkable element 183 gives a remarkable information of non-normal aspect 14, it is possible to conclude to an impossibility to assess the criterion in the given ultrasound image because the remarkable information are contradictory.

As another example, the criterion c1, c2, . . . , ci may be a ventricular asymmetry. It may be evaluated based on two remarkable elements. The first remarkable element 184 relates to the breadth of the right ventricle and the second remarkable element 186 relates to the breadth of the left ventricle. If the first remarkable element 184 is equal to 4 and the second remarkable element 186 is equal to 2, as illustrated in FIG. 8, it is possible to conclude to the presence of a defect in the given ultrasound image due to a ventricular asymmetry. If the first remarkable element 184 is equal to 2 and the second remarkable element 186 is equal to 2, as illustrated in FIG. 9, it is possible to conclude to the absence of a defect in the given ultrasound image because both ventricles have the same size. If the first remarkable element 184 is equal to NA and the second remarkable element 186 is equal to 2, as illustrated in FIG. 10, it is possible to conclude to an impossibility to assess the criterion c1, c2, . . . , ci in the given ultrasound image I1, . . . , In because one ventricle is non observable.

The outputted first scores may take different shapes. For instance, as illustrated in FIG. 12, three mutually exclusive first scores may be defined: an assessability score 31, and a presence score 41 and an absence score 51. The first scores 31, 51, 41 may for instance take a value comprised between 0 and 1. For example, for the assessability score 31, a value approaching 0 may mean that the criterion is assessable. On the contrary, a value approaching 1 means that the criterion c1, c2, . . . , ci is impossible to assess. For the presence score 41, a value approaching 1 means a high probability of presence of a defect. For the absence score 51, a value approaching 1 means a high probability of absence of a defect. The sum of the three first scores 31, 51, 41 may be equal to 1, conveying the fact that the criterion cannot correspond both to a presence of a defect and an absence of a defect or correspond both to an absence of a defect and be impossible to assess or correspond both to a presence of a defect and be impossible to assess.

FIGS. 12 to 14 shows first scores 31, 51, 41 that may be obtained by the transformation module 100 based on the ultrasound image from FIG. 2 to 4. As explained before, two remarkable elements 181, 185 may be extracted from FIG. 2 to 4. A first remarkable element 181 that corresponds to the observability of the region beside the ductal arch and a second remarkable element 185 that corresponds to a vessel in the region beside the ductal arch.

In FIG. 2, the remarkable score 21 associated with the first remarkable element 181 is 0.9 and the remarkable score 25 associated with the second remarkable element 185 is 0.9. The first scores 31, 51, 41 outputted by transformation module 100 corresponding to the fourth vessel beside the ductal arch criterion are illustrated in FIG. 12. The assessability score 31 is equal to 0.1 meaning that the criterion is assessable, the presence score 41 is equal to 0.8, meaning that the probability of presence of a defect is high and the absence score 51 is equal to 0.1, meaning that the probability of absence of a defect is low. The sum of the three first scores 31, 51, 41 is equal to 0.1+0.1+0.8=1.

In FIG. 3, the remarkable score 21 associated with the first remarkable element 181 is 0.9 and the remarkable score 22 associated with the second remarkable element 185 is 0.1. The first scores 31, 51, 41 outputted by transformation module 100 corresponding to the fourth vessel beside the ductal arch criterion are illustrated in FIG. 13. The assessability score 31 is equal to 0.1 meaning that the criterion is assessable, the presence score 41 is equal to 0.1, meaning that the probability of presence of a defect is low and the absence score 51 is equal to 0.8, meaning that the probability of absence of a defect is high. The sum of the three first scores is equal to 0.1+0.8+0.1=1.

In FIG. 4, the remarkable score 21 associated with the first remarkable element 181 is 0.1 and the remarkable score 25 associated with the second remarkable element 185 is 0.1. The first scores 31, 51, 41 outputted by transformation module 100 corresponding to the fourth vessel beside the ductal arch criterion are illustrated in FIG. 14. The assessability score 31 is equal to 0.8 meaning that the criterion is not assessable, the presence score 41 is equal to 0.1 and the absence score 51 is equal to 0.1. Therefore, the probability of presence and absence of a defect are both low because the transformation module 100 is unable to draw a conclusion on this specific ultrasound image. The sum of the three first scores 31, 51, 41 is equal to 0.8+0.1+0.1=1.

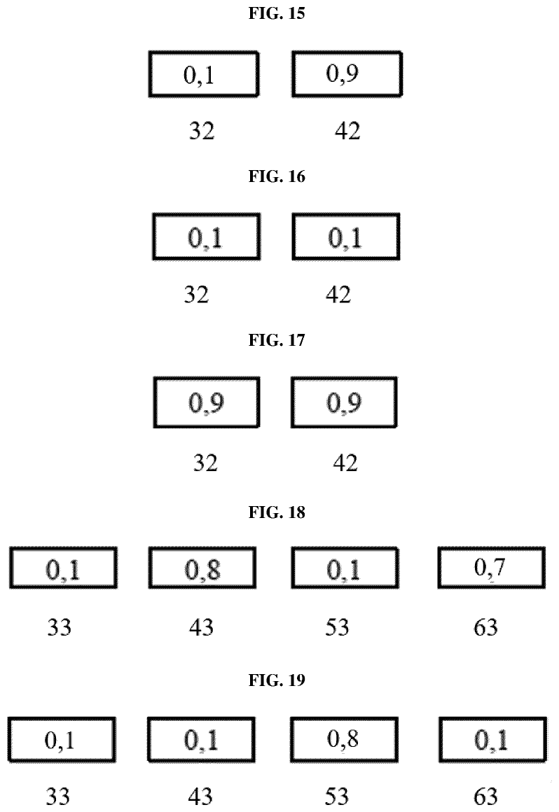

Alternatively, as illustrated in FIG. 15, two first scores 32, 42 may be defined: a defect score 42 including the presence of a defect and the absence of a defect and an assessability score 32, including the impossibility to assess the criterion. The first scores 32, 42 may for instance take a value comprised between 0 and 1. For example, for the assessability score 32, a value approaching 0 may mean that the criterion cannot be correctly assessed. On the contrary, a value approaching 1 means that the criterion can be correctly assessed. Alternatively, a value approaching 0 may convey a possibility to assess the criterion and a value approaching 1 may convey an impossibility to assess the criterion. For the defect score 42, a value approaching 0 means a high probability of presence of a defect with respect to the criterion, while a value approaching 1 means a high probability of absence of a defect with respect to the criterion. Alternatively, a value approaching 0 may mean a high probability of absence of a defect a value approaching 1 means a high probability of presence of a defect.

FIGS. 15 to 17 show first scores 32, 42 that may be obtained by the transformation module 100 based on the ultrasound image from FIG. 5 to 7. As explained before, two remarkable elements 182, 183 may be extracted from FIG. 5 to 7. A first remarkable element 182 that corresponds to a discontinuous septum and a second remarkable element 183 that corresponds to a continuous septum.

In FIG. 5, the remarkable score 22 associated with the first remarkable element 182 is 0.9 and the remarkable score 23 associated with the second remarkable element 183 is 0.1. The first scores 32, 42 outputted by transformation module 100 corresponding to the septal defect criterion are illustrated in FIG. 15. The assessability score 32 is equal to 0.1 meaning that the criterion is assessable, the defect score 42 is equal to 0.9, meaning that the probability of presence of a defect is high.

In FIG. 6, the remarkable score 22 associated with the first remarkable element 182 is 0.1 and the remarkable score 23 associated with the second remarkable element 183 is 0.9. The first scores 32, 42 outputted by transformation module 100 corresponding to the septal defect criterion are illustrated in FIG. 16. The assessability score 32 is equal to 0.1 meaning that the criterion is assessable, the defect score 42 is equal to 0.1, meaning that the probability of absence of a defect is high.

In FIG. 7, the remarkable score 22 associated with the first remarkable element 182 is 0.1 and the remarkable score 23 associated with the second remarkable element 183 is 0.1. The first scores 32, 42 outputted by transformation module 100 corresponding to the septal defect criterion are illustrated in FIG. 17. The assessability score 32 is equal to 0.9 meaning that the criterion is not assessable, the defect score 42 is equal to 0.9 but this value should be interpreted as meaningless to the physician.

Advantageously, a severity score may be included. The severity score may be comprised between 0 and 1 wherein 0 means a low severity and 1 means a high severity of the defect. With reference to FIGS. 18 to 21, it possible to define 4 scores: an assessability score 33, a presence score 43, an absence score 53 and a severity score 63.

FIG. 18 to 21 shows first scores 33, 43, 53, 63 that may be obtained by the transformation module 100 based on the ultrasound imaged from FIG. 8 to 11. As explained before, two remarkable elements 184, 186 may be extracted from FIG. 8 to 11. A first remarkable element 184 that corresponds to the breadth of the right ventricle and a second remarkable element 183 that corresponds to the breadth of the left ventricle.

In FIG. 8, the value 24 associated with the first remarkable element 184 is 4 and the value 26 associated with the second remarkable element 186 is 2. The first scores 33, 43, 53, 63 outputted by transformation module 100 corresponding to the ventricular asymmetry are illustrated in FIG. 18. The assessability score 33 is equal to 0.1 meaning that the criterion is assessable, the presence score 43 is equal to 0.8, meaning that the probability of presence of a defect is high because the ventricles don't have the same breadth in this ultrasound image, the absence score 53 is equal to 0.1, meaning that the probability of absence of a defect is low and the severity score 63 is equal to 0.7, meaning that the asymmetry is quite severe.

In FIG. 9, the value 24 associated with the first remarkable element 184 is 2 and the value 26 associated with the second remarkable element 186 is 2. The first scores 33, 43, 53, 63 outputted by transformation module 100 corresponding to the ventricular asymmetry are illustrated in FIG. 19. The assessability score 33 is equal to 0.1 meaning that the criterion is assessable, the presence score 43 is equal to 0.1, meaning that the probability of presence of a defect is low, the absence score 53 is equal to 0.8, meaning that the probability of absence of a defect is high because both ventricles have the same breadth in this ultrasound image and the severity score 63 is equal to 0.1, meaning that there is no asymmetry.

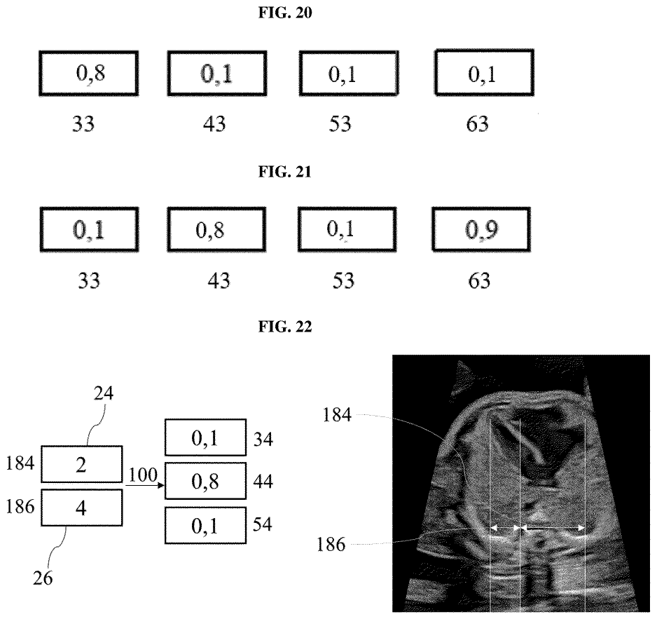

In FIG. 10, the value 24 associated with the first remarkable element 184 is NA and the value 26 associated with the second remarkable element 186 is 2. The first scores 33, 43, 53, 63 outputted by transformation module 100 corresponding to the ventricular asymmetry are illustrated in FIG. 20. The assessability score 33 is equal to 0.8 meaning that the criterion is impossible to assess, the presence score 43, absence score 53 and severity score 63 are equal to 0.1.

In FIG. 11, the value 24 associated with the first remarkable element 184 is 4 and the value 26 associated with the second remarkable element 186 is 1. The first scores 33, 43, 53, 63 outputted by transformation module 100 corresponding to the ventricular asymmetry are illustrated in FIG. 21. The assessability score 33 is equal to 0.1 meaning that the criterion is assessable, the presence score 43 is equal to 0.8, meaning that the probability of presence of a defect is high because there is an asymmetry between the ventricles, the absence score 53 is equal to 0.1, meaning that the probability of absence of a defect is low. The severity score 63 is equal to 0.9 in this ultrasound image. Indeed, compared to FIG. 8, the asymmetry is much more severe than in FIG. 8 because the difference between the ventricles breadth is bigger in FIG. 11 compared to FIG. 8.

The first scores may be obtained using arithmetic operators of linear or non-linear nature such as a CNN or arithmetic means, weighted means, geometric means or harmonic means.

For instance, the transformation module 100 may be configured to take as input a matrix with two dimensions 2×3. The 2 lines correspond to two remarkable elements. The first column corresponds to a remarkable score associated to each remarkable element. The remarkable score comprises observability/unobservability information. The remarkable score is comprised between 0 and 1, and the last two columns correspond to location information in the form of coordinates of the remarkable elements.

For instance, the transformation module 100 may be configured to output a matrix with two dimensions 1×2. The line corresponds to a first criterion and the two columns to two first scores. One first score may be extracted as the mean of the observability scores of the two remarkable elements. The second first score may be extracted as the division between the two breadths measurement calculated from the coordinates of the remarkable elements, followed by a normalization operation to obtain a value equal to 0 if the ratio is close to reference value, and value equal to 1 if the ratio is far from reference value, power the mean of the observability scores.

As another example, the transformation module 100 may be configured to output a matrix with two dimensions 1×3. The line corresponds to a first criterion and the three columns to three remarkable scores comprising respectively observability/unobservability, normality/non-normality and abnormality/non-abnormality information. The first scores can be extracted through a multi-class softmax classification which outputs three mutually exclusive classification confidences.

Because the ultrasound images are only partial views of the organ, the outputted first scores may be biased, for instance because of the ultrasound image angle. The temporal module 200 is configured to aggregate the information extracted from all the ultrasound images I1, . . . , In, to output global information representative of the actual and unbiased health status of the organ.

As illustrated in FIG. 1, the lists of remarkable elements e1, e2, e3, . . . , ek and/or the lists of first criteria c1, c2, . . . , ci obtained for each ultrasound image I1, . . . , In are fed to a temporal module 200. The temporal module 200 is configured to aggregate the first scores 30, 40 outputted by the transformation module 100 for each image of the set of ultrasound images I1, . . . , In and to output a list of global criteria C1, C2, . . . , Cj, wherein each criterion of the list of global criteria C1, C2, . . . , Cj, is associated with at least two global scores 70, 80 corresponding to the behavior of a corresponding criterion of the list of first criteria c1, c2, . . . , ci across the whole set of ultrasound images I1, . . . , In.

The global scores are derived from an analysis of the remarkable elements across at least a sub-set of ultrasound images comprising at least two ultrasound images or, preferably, the whole set of ultrasound images. Indeed, one same remarkable element may or may not be observed in different ultrasound images depending on the viewpoint of the ultrasound image and on the quality of the ultrasound image. If one remarkable element is consistently associated with the same remarkable information, it may indicate a high probability of presence or an absence of a defect. On the other hand, if the remarkable element appears in different locations, presents different dimensions or is difficult to identify in some images, this may either indicate that the set of ultrasound images is not qualitative enough to draw conclusions on the global criterion, or it may indicate that there is a problem with the organ with respect to the global criterion, such as a lesion or an abnormal growth.

For instance, a global criterion may be a global ventricular asymmetry. The global asymmetry may be evaluated over several ultrasound images I1, . . . , In. Indeed, the heart being a dynamic organ, its shape changes periodically through time. Throughout this change, the visual aspect ratio of globally asymmetrical ventricles can seem normal sporadically on some ultrasound images I1, . . . , In. Inversely, the visual aspect ratio of globally symmetrical ventricles can seem abnormally asymmetrical sporadically on other ultrasound images I1, . . . , In. The global scores 70, 80 take into account first scores determined over a large number of images I1, . . . , In. Therefore, they are more representative of the actual ratio between the ventricles.

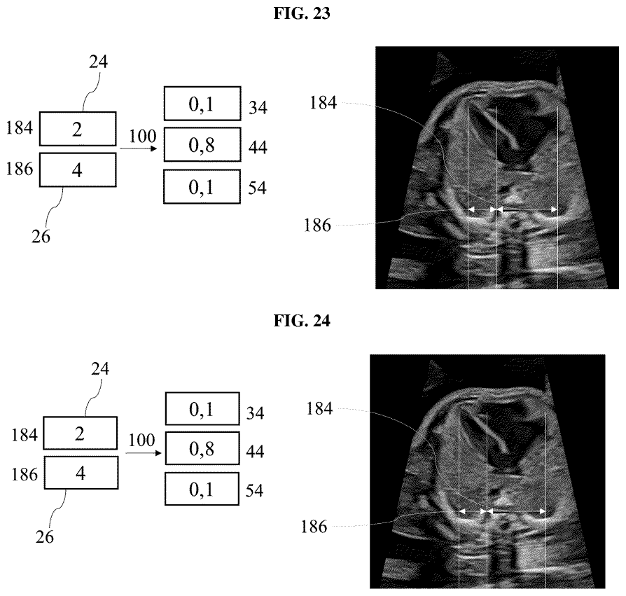

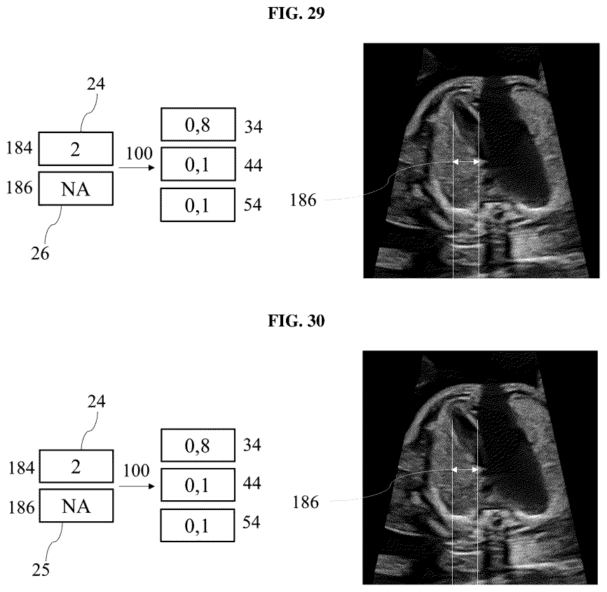

As an example, 3 ultrasound images may be used as illustrated in FIGS. 22 to 24, FIGS. 25 to 27 or FIGS. 28 to 30. In these ultrasound images, remarkable elements 184 and 186 have been extracted. These remarkable elements 184, 186 respectively correspond to the breadth of the right and left ventricles. The transformation module 100 outputted three mutually exclusive first scores 34, 44, 54 corresponding to an assessability score 34, a presence score 44 and an absence score 54.

In the three ultrasound images from FIGS. 22 to 24, there is an asymmetry between the ventricles and the presence score 44 is equal to 0.8, while the assessability score 34 and the absence score 54 are equal to 0.1. Therefore, the global scores will also conclude to the global presence of a defect because all ultrasound images contain the same first information of presence of a defect.

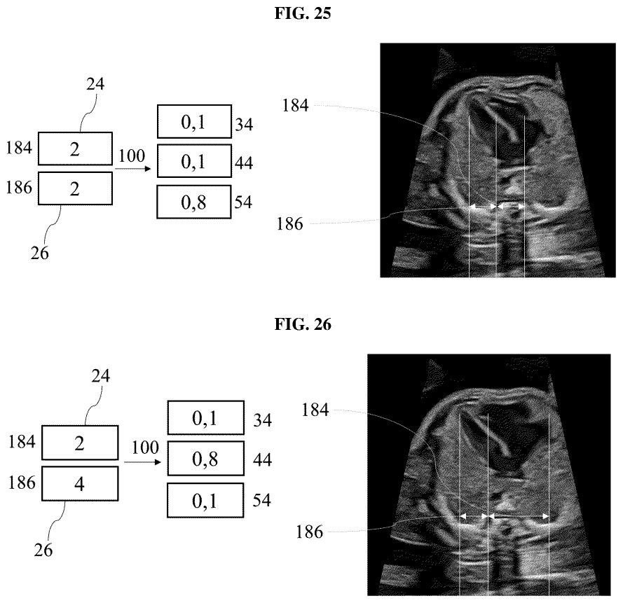

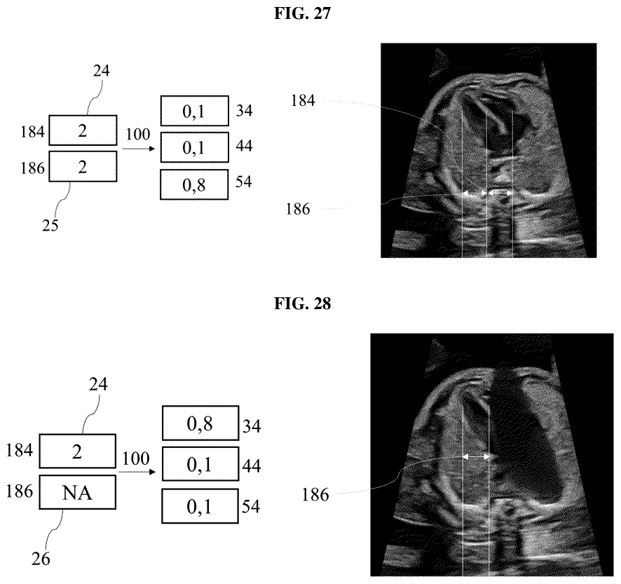

In the three ultrasound images from FIGS. 25 to 27, there is no asymmetry between the ventricles in FIGS. 25 and 27. The absence score 44 is equal to 0.8 in these two figures, while the assessability score 34 and the presence score 54 are equal to 0.1. However, there is an asymmetry in FIG. 26 and the presence score 44 is equal to 0.8, while the assessability score 34 and the absence score 54 are equal to 0.1. In this case, the global scores will conclude to the global absence of a defect because a majority of the ultrasound images contain the same first information of absence of a defect.

In the three ultrasound images from FIGS. 28 to 30, there is a shadow over the right ventricle and the remarkable element 186 is impossible to measure. Therefore, the assessability score 34 is equal to 0.8, while the presence score 44 and the absence score 54 are equal to 0.1. In this case, the global scores will conclude to the global impossibility to assess the set of ultrasound images because all the ultrasound images contain the same first information of impossibility to assess the criterion.

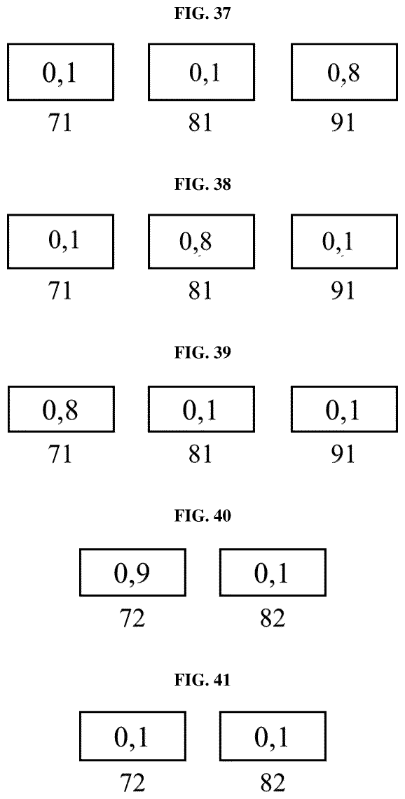

As illustrated in FIG. 37 to 39, the global scores may correspond to three mutually exclusive global scores 71, 81, 91: a global assessability score 71, a global absence score 81 and a global presence score 91. The global scores 71, 81, 91 may for instance take a value comprised between 0 and 1. For example, for the global assessability score 71, a value approaching 0 may mean that the global criterion is assessable. On the contrary, a value approaching 1 means that the global criterion C1, C2, . . . , Cj is impossible to assess. For the absence score 81, a value approaching 1 means a global high probability of absence of a defect. For the presence aspect score 91, a value approaching 1 means a global high probability of presence of a defect. The sum of the three global scores 71, 81, 91 may be equal to 1, conveying the fact that the global criterion cannot correspond both to a global presence of a defect and a global absence of a defect or correspond both to a global absence of a defect and be impossible to assess or correspond both to a global presence of a defect and be impossible to assess.

The global scores that may be obtained using the temporal module 200 based on the ultrasound images from FIGS. 22 to 24 are illustrated in FIG. 37. The global assessability score 71 is equal to 0.1 meaning that the global criterion is assessable, the global absence score 81 is equal to 0.1, meaning that the global probability of absence of a defect is low and the global presence score 91 is equal to 0.8, meaning that the global probability of presence of a defect is high. The sum of the three global scores 71, 81, 91 is equal to 0.1+0.1+0.8=1.

The global scores that may be obtained using the temporal module 200 based on the ultrasound images from FIGS. 25 to 27 are illustrated in FIG. 38. The global assessability score 71 is equal to 0.1 meaning that the global criterion is assessable, the global absence score 81 is equal to 0.8, meaning that the global probability of absence of a defect is high and the global presence score 91 is equal to 0.1, meaning that the global probability of presence of a defect is low. The sum of the three global scores 71, 81, 91 is equal to 0.1+0.8+0.1=1.

The global scores that may be obtained using the temporal module 200 based on the ultrasound images from FIGS. 28 to 30 are illustrated in FIG. 39. The global assessability score 71 is equal to 0.8 meaning that the global criterion is impossible to assess, the global absence score 81 and the global presence score 91 are equal to 0.1. The sum of the three global scores 71, 81, 91 is equal to 0.8+0.1+0.1=1.

Alternatively, two global scores may be defined: a global defect score 72 including the global presence of a defect and the global absence of a defect, and a global assessability score 82, including the global impossibility to assess the global criterion. The global scores 72, 82 may for instance take a value comprised between 0 and 1. For example, for the global assessability score 82, a value approaching 0 may mean that it is impossible to assess the global criterion. On the contrary, a value approaching 1 means that it is possible to assess the global criterion. For the global defect score 72, a value approaching 0 means a high probability of global presence of a defect, while a value approaching 1 means a high probability of global absence of a defect.

For instance, a global criterion C1, C2, . . . , Cj may be a static mitral valve. In that specific case, the criterion cannot be assessed using a single ultrasound image because it is a temporal criterion. Indeed, a valve being a dynamic element, it periodically opens and closes through time. Therefore, it is meaningless to try to determine first criterion c1, c2, . . . , ci scores on the mitral valve using the transformation module 100 as some ultrasound images may show the valve closed and some other images may show the valve opened. The temporal module 200 is configured to aggregate these findings to verify if the valve is regularly opening or closing. In that case, the temporal module directly takes the remarkable information associated with at least one remarkable element as an input.

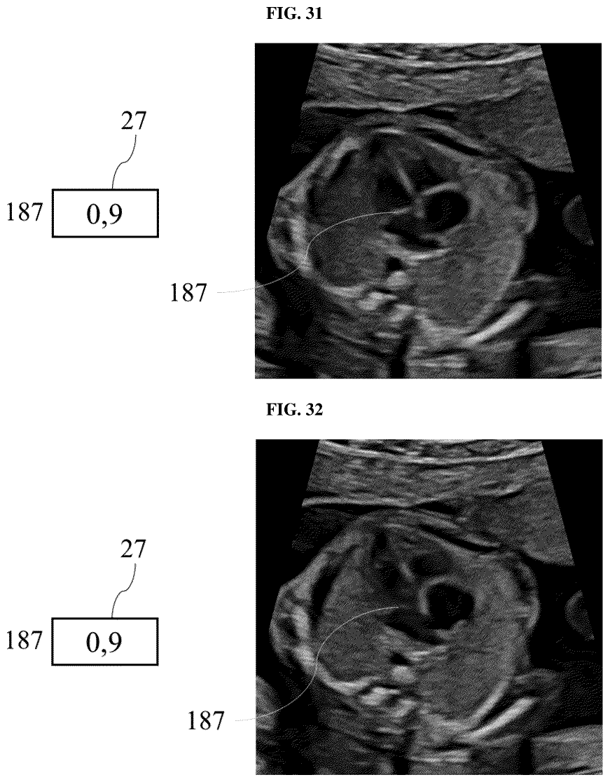

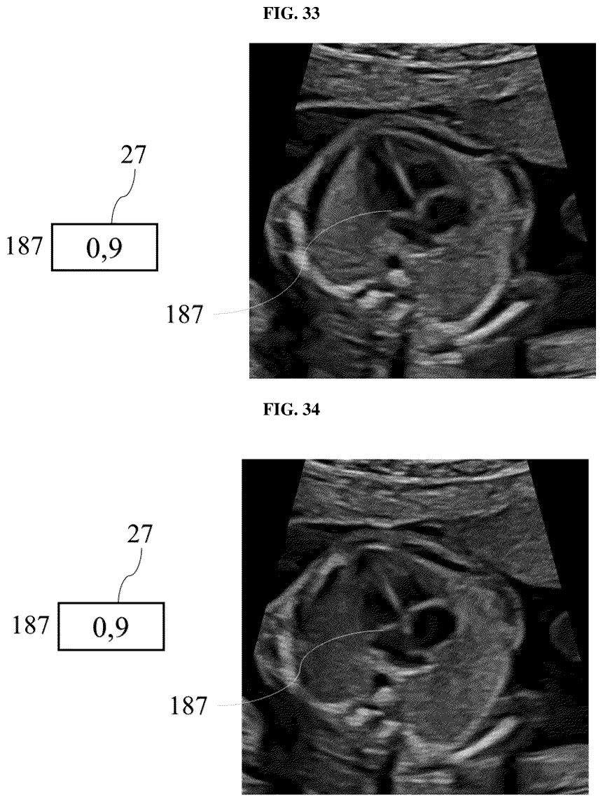

Accordingly, this global criterion C1, C2, . . . , Cj may be assessed directly using remarkable element 187 as illustrated in FIGS. 31 to 33 and 34 to 36. Alternatively, the remarkable information contained in the remarkable element 187 may be stored by the transformation module 100 in the list of first criteria c1, c2, . . . , ci of the ultrasound image but the value will be meaningless to the physician as it does not correspond to a score.

In FIGS. 31 to 33, the static mitral valve is always closed. The remarkable element 187 is associated with a remarkable score 27 comprised between 0 and 1, wherein a value approaching 0 means that the mitral valve is well observable and is in a closed state, and a value approaching 1 means that the mitral valve is not observable and in an open state. In FIGS. 31 to 33, the remarkable score 27 is always equal to 0.9.

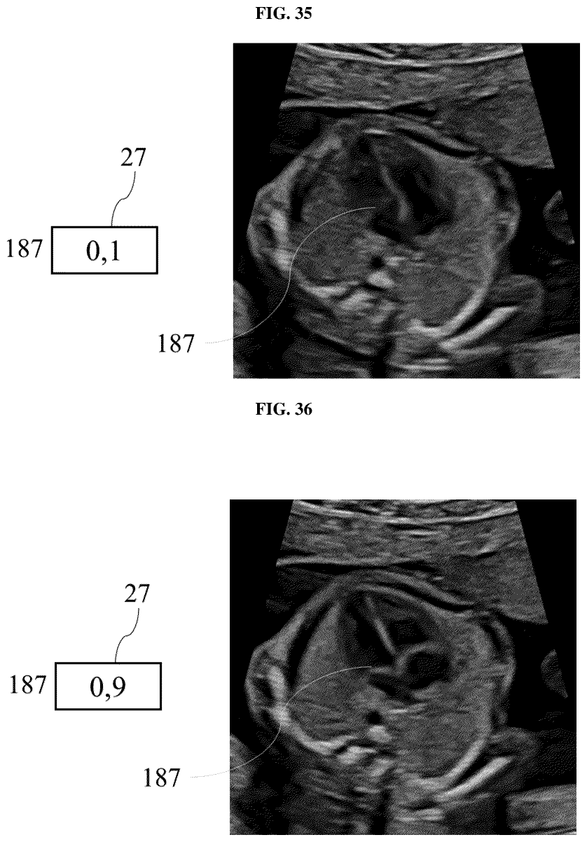

In FIGS. 34 to 36 the static mitral valve is periodically opening and closing. As a consequence, the remarkable score 27 is equal to 0.9 in FIGS. 34 and 36 and the remarkable score 27 is equal to 0.1 in FIG. 35.

The two global scores 72, 82 associated with FIGS. 31 to 33 are illustrated in FIG. 40. The global defect score 72 is equal to 0.9 and the global assessability score 82 is equal to 0.1, thus conveying that the static mitral valve is well observable but always closed.

The two global scores 72, 82 associated with FIGS. 34 to 36 are illustrated in FIG. 41. The global defect score 72 is equal to 0.1 and the global assessability score 82 is equal to 0.1, thus conveying that the static mitral valve is well observable and periodically opening and closing.

To output the global scores, the temporal module 200 implements a feed-forward network of arithmetic operators of linear or non-linear nature such as a 1-dimensional CNN. Such CNN processes simultaneously the lists of first criteria c1, c2, . . . , ci outputted by the transformation module 100 to outputs the list of global criteria. Alternatively, the temporal module 200 may be configured to implement a recurrent CNN configured to iteratively process each list of first criteria c1, c2, . . . , ci to output the list of global criteria C1, C2,. . . , Cj.

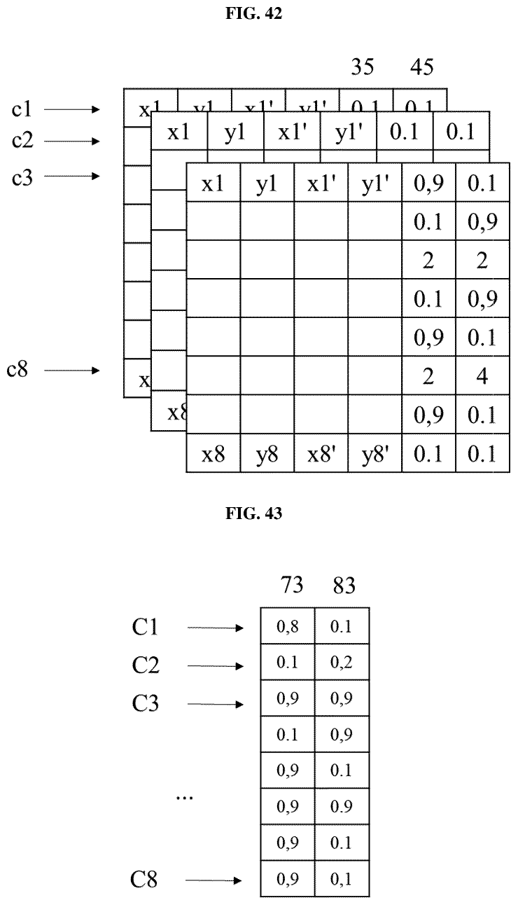

For instance, the CNN can be configured to take as input a matrix with three dimensions 6×8×50 such as illustrated in FIG. 42. The 8 lines correspond to the first criteria c1-c8. The first four column correspond to location information in the form of coordinates x1,. . . , x8, y1, . . . , y8, x1′, . . . , x8′, y1′, . . . , y8′ of a bonding box corresponding to the position in the ultrasound image of the remarkable elements used to verify the criterion. The fifth and sixth columns corresponds to the at least two first scores 35, 45 determined by the transformation module 100. The third dimension corresponds to the number of ultrasound image I1, . . . , In, which in this case is equal to 50.

One or more convolutional layers may be successively applied to this matrix. The layers are defined by N kernels of size T followed by a non-linear activation function.

The CNN may output a matrix with two dimensions 2×8 such as illustrated in FIG. 43. The 8 lines correspond to the global criteria C1-C8. The two columns correspond to the at least two global scores 73, 83.

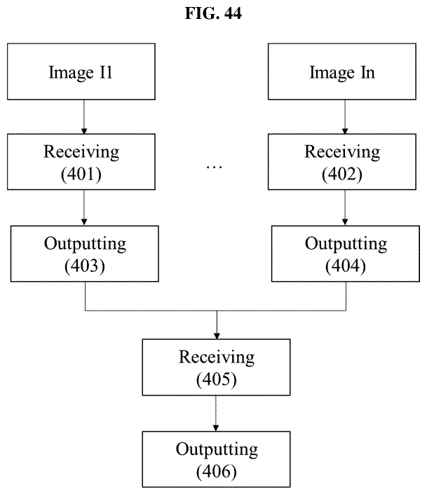

According to another aspect, the invention relates to a method implemented by the device 1000, as illustrated on FIG. 44. For each ultrasound image of a set of ultrasound images I1, . . . , In, the method carries out step 401, 402 of receiving a list of remarkable elements and step 403, 404 of outputting a list of first criteria with at least two first scores per criterion of the list of first criteria. These steps may be carried out successively or simultaneously. Once all the lists of first criteria have been generated for all the ultrasound images, the method carries out step 405 of receiving all the outputted lists of first criteria and step 406 of outputting a list of global criteria with at least two global scores per criterion of the list of global criteria.

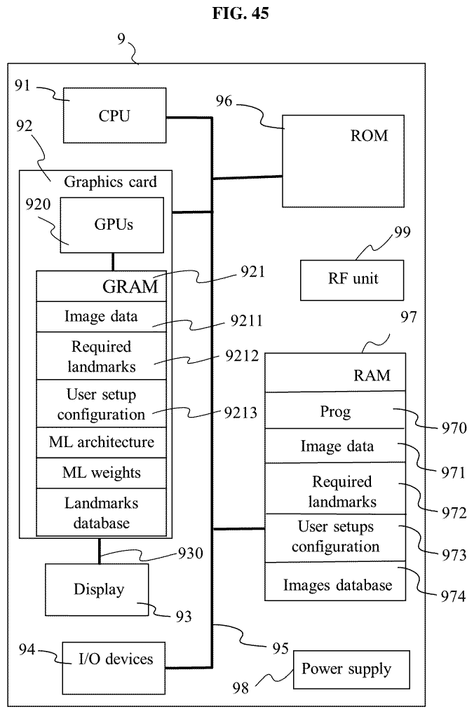

A particular apparatus 9, visible on FIG. 45, is embodying the device 1000 for guiding a user in ultrasound assessment of an organ described above. It corresponds, for example, to a workstation, a laptop, a tablet, a smartphone, or a head-mounted display (HMD).

That apparatus 9 is suited for guiding a user in ultrasound assessment of an organ. comprises the following elements, connected to each other by a bus 95 of addresses and data that also transports a clock signal:
a microprocessor 91 (or CPU);
a graphics card 92 comprising several Graphical Processing Units (or GPUs) 920 and a Graphical Random Access Memory (GRAM) 921; the GPUs are quite suited to image processing by e.g. the CNN block, due to their highly parallel structure;
a non-volatile memory of ROM type 96;
a RAM 97;
one or several I/O (Input/Output) devices 94 such as for example a keyboard, a mouse, a trackball, a webcam; other modes for introduction of commands such as for example vocal recognition are also possible;
a power source 98; and
a radiofrequency unit 99.

According to a variant, the power supply 98 is external to the apparatus 9.

The apparatus 9 also comprises a display device 93 of display screen type directly connected to the graphics card 92 to display synthesized images calculated and composed the graphics card. The use of a dedicated bus to connect the display device 93 to the graphics card 92 offers the advantage of having much greater data transmission bitrates and thus reducing the latency time for the displaying of images composed by the graphics card, for example for identifying and localizing the landmarks. According to a variant, a display device is external to apparatus 9 and is connected thereto by a cable or wirelessly for transmitting the display signals. The apparatus 9, for example through the graphics card 92, comprises an interface for transmission or connection adapted to transmit a display signal to an external display means such as for example an LCD or plasma screen a video-projector. In this respect, the RF unit 99 can be used for wireless transmissions.

It is noted that the word “register” used hereinafter in the description of memories 97 and 921 can designate in each of the memories mentioned, a memory zone of low capacity (some binary data) as well as a memory zone of large capacity (enabling a whole program to be stored or all or part of the data representative of data calculated or to be displayed). Also, the registers represented for the RAM 97 and the GRAM 921 can be arranged and constituted in any manner, and each of them does not necessarily correspond to adjacent memory locations and can be distributed otherwise (which covers notably the situation in which one register includes several smaller registers).

When switched-on, the microprocessor 91 loads and executes the instructions of the program contained in the RAM 97.

As will be understood by a skilled person, the presence of the graphics card 92 is not mandatory, and can be replaced with entire CPU processing and/or simpler visualization implementations.

In variant modes, the apparatus 9 may include only the functionalities of the device 1, and not the learning capacities of the device for training. In addition, the device 1 and/or the device for training may be implemented differently than a standalone software, and an apparatus or set of apparatus comprising only parts of the apparatus 9 may be exploited through an API call or via a cloud interface.

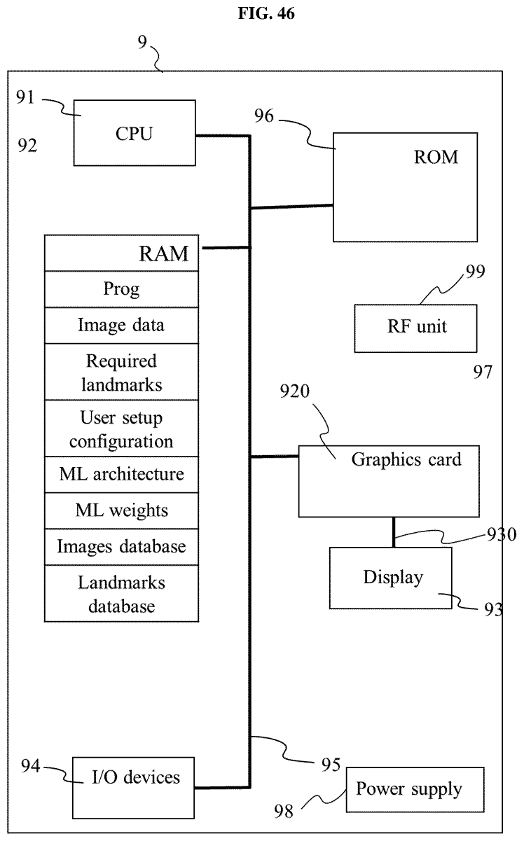

FIG. 46 shows an alternative example for the apparatus 9 comprising an appropriate GPU (one that can be used for ML calculation). For example, a simple video card may be integrated into processor. It has no CUDA technology able to paralyze numerical computations.

1. A device for guiding a user in ultrasound assessment of an organ to perform an evaluation of said organ during a medical examination, said ultrasound assessment being based on at least two ultrasound images resulting from at least a partial reflection of ultrasound waves by said organ, said device comprising at least one processor configured to:
for each ultrasound image:
receive as an input a list of remarkable elements associated to the ultrasound image wherein each remarkable element is associated with at least one remarkable information among:
an observability of the remarkable element or an unobservability of the remarkable element,
an abnormal aspect of the remarkable element or a non-abnormal aspect of the remarkable element,
a normal aspect of the remarkable element, or a non-normal aspect of the remarkable element, and
a measurement performed on the remarkable element,
generate and output a list of first criteria indicative of a health status of the organ, wherein each first criterion of the list of first criteria is associated with at least two first scores, each first score being generated using said at least one remarkable information associated to the at least one remarkable element, said at least two first scores being such that a combination of the at least two first scores provides first information on an impossibility to assess the first criterion, a presence of a defect and an absence of a defect,
generate and output a list of global criteria indicative of a health status of the organ assessed over said at least two ultrasound images, wherein each global criterion of the list of global criteria is associated with at least two global scores, said at least two global scores being such that a combination of the at least two global scores provides a global information on a global impossibility to assess the global criterion, a global presence of a defect and a global absence of a defect, wherein the list of global criteria is generated using a feed-forward network of arithmetic operators of linear or non-linear nature which maps at least two of said generated lists of first criteria, and/or at least two lists of remarkable elements relative to the at least two ultrasound images from which said at least two lists of first criteria have been generated, to the at least two global scores.

2. The device according to claim 1, wherein at least one global criterion is a temporal criterion indicative of a defect in a periodic evolution of the organ and the at least two global scores are determined based on the evolution in time of the at least one remarkable element across the at least two ultrasound images.

3. The device according to claim 1, wherein each first criterion of the list of first criteria is further associated with a first severity score representative of a magnitude of the defect.

4. The device according to claim 1, wherein each global criterion of the list of global criteria is further associated with a global severity score representative of a magnitude of the defect assessed on the at least two ultrasound images.

5. The device according to claim 1, wherein the at least one remarkable information is comprised in at least one remarkable score.

6. The device according to claim 1, wherein each first criterion of the list of first criteria is associated with three mutually exclusive first scores, including:
an assessability score, representative of the impossibility to assess the first criterion,
a presence score, representative of the presence of a defect, and
an absence score, representative of the absence of a defect.

7. The device according to claim 1, wherein each first criterion of the list of first criteria is associated with two first scores:
a defect score representative of the presence of a defect and the absence of a defect, said defect score being comprised between a lower score bound and an upper score bound, the lower score bound being representative of a high probability of an absence of a defect and the upper score bound being representative of a high probability of a presence of a defect, and
an assessability score, being representative of the impossibility to assess the first criterion.

8. The device according to claim 1, wherein each global criterion of the list of global criteria is associated with two global scores:
a global defect score representative of the global absence of a defect and the global presence of a defect, said global defect score being comprised between a global lower score bound and a global upper score bound, the global lower score bound being representative of a high probability of an absence of a defect across the at least two ultrasound images and the upper score bound being representative of a high probability of a presence of a defect across the at least two ultrasound images, and
a global assessability score representative of the global impossibility to assess the global criterion.

9. The device according to claim 1, wherein the generation of the list of global criteria comprises implementing a 1-dimensional convolutional neural network configured to simultaneously process the outputted lists of first criteria and/or the lists of remarkable elements relative to the at least two ultrasound images.

10. The device according to claim 1, wherein the generation of the list of global criteria comprises implementing a recurrent convolutional neural network configured to iteratively process the outputted lists of first criteria and/or the lists of remarkable elements relative to the at least two ultrasound images.

11. A computer-implemented method for guiding a user in ultrasound assessment of an organ to perform a diagnosis or screening evaluation of said organ during a medical examination, said ultrasound assessment being based on at least two ultrasound image resulting from at least a partial reflection of ultrasound waves by said organ, said method comprising:
for each ultrasound image:
receiving as an input, for each ultrasound image of the at least two ultrasound images, a list of remarkable elements associated to the ultrasound image, wherein each remarkable element is associated with at least one remarkable information among:
an observability of the remarkable element or an unobservability of the remarkable element, p3 an abnormal aspect of the remarkable element or a non-abnormal aspect of the remarkable element
a normal aspect of the remarkable element or a non-normal aspect of the remarkable element, and
a measurement performed on the remarkable element,
generating and outputting a list of first criteria indicative of a health status of the organ, wherein each first criterion of the list of first criteria is associated with at least two first scores, each first score being generated using said at least one remarkable information associated to the at least one remarkable element, said at least two first scores being such that a combination of the at least two first scores provides information on an impossibility to assess the first criterion, a presence of a defect, and an absence of a defect,
generating and outputting a list of global criteria indicative of a health status of the organ assessed over said at least two ultrasound images, wherein each global criterion of the list of global criteria, said at least two global scores being such that a combination of the at least two global scores provides information on a global impossibility to assess the global criterion, a global presence of a defect and a global absence of a defect, wherein the list of global criteria is generated using a feed-forward network of arithmetic operators of linear or non-linear nature which maps at least two of said generated lists of first criteria, and/or at least two lists of remarkable elements relative to the at least two ultrasound images from which said at least two lists of first criteria have been generated, to the at least two global scores.

12. (canceled)

13. A non-transitory_computer-readable storage medium comprising instructions which, when executed by a computer, cause the computer to carry out the method for guiding a user in ultrasound assessment of an organ according to claim 11.
